(19) United States
(12) Patent Application Publication
(10) Pub. No.: US 2024/0252184 A1
(43) Pub. Date: Aug. 1, 2024

(54) REAMER

(71) Applicant: ANIKA THERAPEUTICS, INC., Bedford, MA (US)

(72) Inventor: Steven W. Ek, Durham, NH (US)

(21) Appl. No.: 18/430,154

(22) Filed: Feb. 1, 2024

Related U.S. Application Data
(60) Provisional application No. 63/482,773, filed on Feb. 1, 2023.

(51) Int. Cl. A61B 17/16

(52) U.S. Cl. CPC A61B 17/164; A61B 17/1668

(57) ABSTRACT

Described herein are reverse shoulder devices and related systems, methods, and kits useful in orthopedic surgery. In some embodiments, a reamer is described having a handle, a shank and a cutting body. In some embodiments, the cutting body has a threaded region and a tapered cutting region. In some embodiments, the cutting body is configured to compact bone particles against the inner surface of a bone bore while reaming the bone.

### CROSS-REFERENCE TO RELATED APPLICATIONS

This application claims the benefit of U.S. Provisional Patent Application No. 63/482,773, filed Feb. 1, 2023, the entire contents of which are incorporated by reference herein.

### TECHNICAL FIELD

The present disclosure is related to devices, systems, methods, and kits for reverse orthopedic surgery. Specifically, the disclosure is directed to a reamer tool and related instruments for performing surgery to implant orthopedic implants.

### BACKGROUND

Articular bones in the human body have articular cartilage covering the ends of the bone, particularly where one bone interfaces with another bone such as in a joint. Articular cartilage is smooth, load bearing, and lubricious, allowing one bone to slip past another bone while maintaining strength during movement. When a bone is injured, this articular cartilage may be damaged. Furthermore, as the body ages articular cartilage can naturally break down, causing bone to rub on bone leading to pain for the patient, reduced mobility, and osteoarthritis.

In some cases, the extent of damage necessitates repair to the cartilage using one or more implants. When replacing a joint with an implant it is important to choose an implant that is mechanically stable and allows for full mobility and movement in the joint.

The implant should be designed to maximize the patient's comfort, minimize damage to surrounding areas, minimize potential further injury, maximize the functional life of the implant, and be easy to install. The longer the amount of time a patient spends in surgery, the greater risk there is for complications for the patient and increased recovery time. Therefore, there is a need for tools, surgical techniques, and systems to reduce overall surgery time.

Joint replacement surgery can require revisions to the placement of an implant or may necessitate an entirely new implant be placed. When placing a new implant or moving an existing implant, the amount of healthy bone for placement of the implant will impact the ease and speed of placement surgery. Without sufficient healthy bone, an implant cannot be placed. Therefore, there is a need for tools, systems, and methods to preserve as much healthy bone as possible when performing orthopedic surgeries.

It is important to preserve as much bone as possible when performing a joint replacement surgery. Healthy bone serves as a foundation for implants placed within a portion of the bone. Sufficient strength of the surrounding bone is necessary to support fixation of the implant and prevent undesirable moving or decoupling from the bone.

Traditional joint replacement involves removing or disposing a bore into the upper condyle or a portion of the upper condyle of an articulating bone. An implant may then be placed into the bone with a stem and cemented into place.

The systems methods and devices described herein are directed towards preserving healthy bone and reducing surgery time for orthopedic surgeries. In some embodiments, the devices, systems, and methods described herein are directed towards reverse shoulder surgery.

### SUMMARY

In one aspect, embodiments described herein relate to systems for performing an orthopedic surgery on a patient. In some embodiments, a reamer is described including a handle, a shank, and a cutting body for performing an orthopedic surgery on a subject. The reamer comprises a handle configured to be gripped by a surgeon. The cutting body comprising a threaded region configured to compact bone particles within a bone bore and a tapered region configured to cut a bore into bone. The shank connects the handle to the cutting body.

In another aspect, embodiments described herein relate to a reamer for performing an orthopedic surgery on a subject. In some embodiments, the reamer includes a handle configured to be gripped by a user and a cutting body coupled to the handle and having a threaded region configured to compact bone particles within a bone bore, and a tapered region configured to cut a bore into bone. In some embodiments, the reamer includes a shank configured to couple the handle to the cutting body. In some embodiments, the shank is releasably coupled with the cutting body. In some embodiments, the reamer has an annular disk extending radially from the shank. In some embodiments, the annular disk is spaced apart from the tapered region so as to correspond to a maximum depth of the tapered region within the bore when the annular disk interfaces with an outer surface of the bone. In some embodiments, the reamer includes a neck region extending between the shank and the cutting body, the neck region tapering radially inwards from a wide end interfacing with the cutting body to a narrow end interfacing with the shank. In some embodiments, the tapered region includes a first protrusion extending from an external surface of the tapered region. In some embodiments, the reamer includes a second protrusion extending from the external surface of the tapered region. In some embodiments, the tapered region includes a first cutting channel. In some embodiments, the reamer includes an opening extending through a longitudinal axis of the cutting body. In some embodiments, the threaded region further includes a first notch extending longitudinally between a proximal and distal end of the cutting body and at least partially recessed into the threaded region. In some embodiments, the threaded region includes a second notch extending longitudinally between the proximal and distal end of the cutting body and at least partially recessed into the threaded region. In some embodiments, the threaded region includes one or more threads angled 0°, 5°, 10°, 15°, 20°, 25°, or 30°, relative to a circumference axis of the threaded region.

In another aspect, embodiments described herein relate to a method of forming a bore in a bone. The method includes reaming a portion of the bone to form the bore within the bone, compacting a plurality of bone particles against an inner surface of the bore by rotating a cutting body comprising a threaded region and a tapered region, wherein the threaded region comprises one or more threads configured to compact the plurality of bone particles about the inner surface of the bone bore, and removing the cutting body from the bone bore. In some embodiments, one or more threads are angled 0°, 5°, 10°, 15°, 20°, 25°, or 30°, relative to a circumference axis of the threaded region. In some embodiments, the cutting body is releasably coupled to a shank. In some embodiments, the method includes reaming the bore to achieve a final depth of the tapered region within the bore, such that an annular disk extending radially from the shank interfaces with an outer surface of the bone. In some embodiments, the threaded region further comprises a first notch extending longitudinally between a proximal and distal end of the cutting body and at least partially recessed into the threaded region. In some embodiments, the threaded region further comprises a second notch extending longitudinally between the proximal and distal end of the cutting body and at least partially recessed into the threaded region. In some embodiments, the method includes passing an opening extending through a longitudinal axis of the cutting body over a surgical guide pin disposed in the bone.

### BRIEF DESCRIPTION OF THE DRAWINGS

FIG. 1 illustrates an exploded view of a reverse shoulder system with a humeral implant and a glenoid implant, according to an embodiment herein.

FIGS. 2A-W illustrate tools, systems, and methods used in relation to a humeral implant in a reverse shoulder surgery, according to an embodiment herein.

FIG. 3A illustrates a reamer having a shank and a cutting body according to certain embodiments described herein. FIG. 3B and 3C illustrate various angles of the cutting body according to embodiments described herein.

### DETAILED DESCRIPTION

This disclosure presents various systems, components, and methods related to disposing a bore into a bone. In some embodiments. this disclosure presents various systems, components, and methods related to a reverse shoulder surgery. Each of the systems, components, and methods disclosed herein provides one or more advantages over traditional systems, components, and methods. Various embodiments of the reverse shoulder surgery devices, systems, components, and methods are disclosed herein.

As used herein, the term “handle” refers to any protuberance or recess from a surface which may be gripped with a hand, tool, or device.

As used herein, the terms “proximal” and “distal” refer to the proximal and distal directions relative to the user of the component. For example, if a surgeon is holding a tool used to place reverse shoulder implant component, the distal and proximal portions of the tool are relative to the surgeon holding the tool. The term “proximal” refers to an area, surface, or point situated nearer to the center of the body. The term “distal” refers to an area, surface, or point situated further from the center of the body.

As used herein, the term “about” means within ±10% of the value it modifies. For example, “about 1” means “0.9 to 1.1”, “about 2%” means” 1.8% to 2.2%“, “about 2% to 3%” means” 1.8% to 3.3%”, and “about 3% to about 4%” means “2.7% to 4.4%.” Unless otherwise clear from the context, all numerical values provided herein are modified by the term “about”.

Orthopedic implants, such as glenoid or humoral implants, may be placed at least partially into bone to replace an articulating surfaces of a shoulder joint. Specifically, the glenoid and/or humeral implants are disposed at least partially within the humorous and/or glenoid portion of the scapula bones.

Disclosed herein, in some aspects, are systems and methods for performing an orthopedic surgery. In some embodiments, tools, components, systems, and methods are used to perform a reverse shoulder surgery. In some embodiments, the system comprises a humeral implant and/or a glenoid implant. In some embodiments, the humeral implant comprises a tray configured to detachably couple to a humeral implant site of a humerus, wherein the tray is configured to receive a liner for receiving a head portion of an implant. In some embodiments, the humeral implant includes a stem at least partially inserted within the humerus. In some embodiments, the stem, at a proximal portion thereof, includes an opening to a cavity within the stem configured to receive a tray base portion. In some embodiments, an adapter is configured to be received by the cavity and is selectively sized so as to interface between an inner surface of the stem defining the cavity and the tray base portion. In some embodiments, the adapter is sized so as to eliminate or reduce unfilled spacing between at least a portion of the cavity inner surface and the tray base portion, thereby eliminating or reducing a potential for movement of the tray and/or stem about the humeral implant site. In some embodiments, the adapter is sized using an offset tool, configured to measure a distance between an opening at the humeral implant site and a bottom inner surface of the cavity.

In some embodiments, aside from the coupling of the stem to the adapter and/or tray, and at least partially due to a friction fit of the stem within the humerus bone, the stem is not otherwise secured to the humerus via additional securing means (e.g., there is no use of bone cement, threading of the stem to the humerus bone, additional screws, etc.). Accordingly in some embodiments, the humeral implant provides only one means of fixation to the humerus (via the tray engagement with the humerus implant site as described herein). In some embodiments, such single means of fixation provides flexibility for the installation and/or arrangement of the humerus implant, as well as flexibility with the interaction with the glenoid implant.

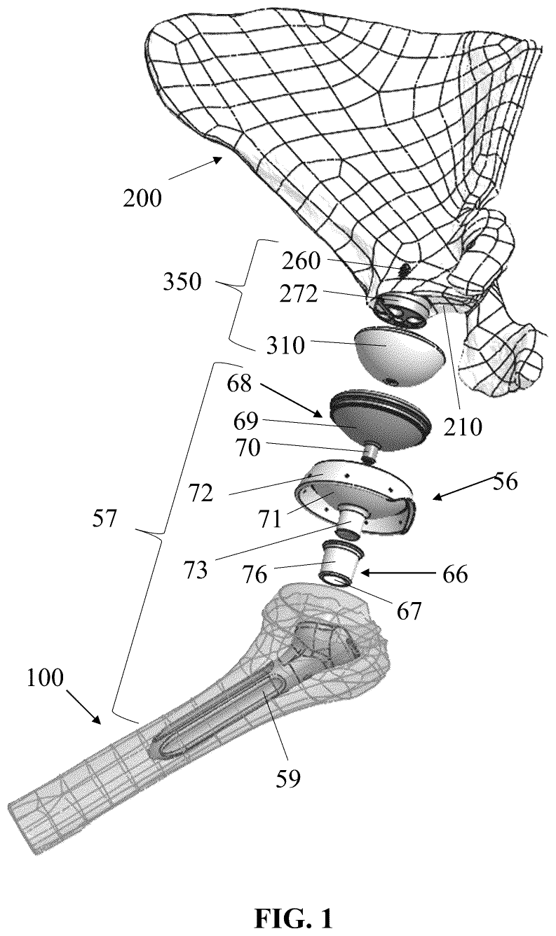

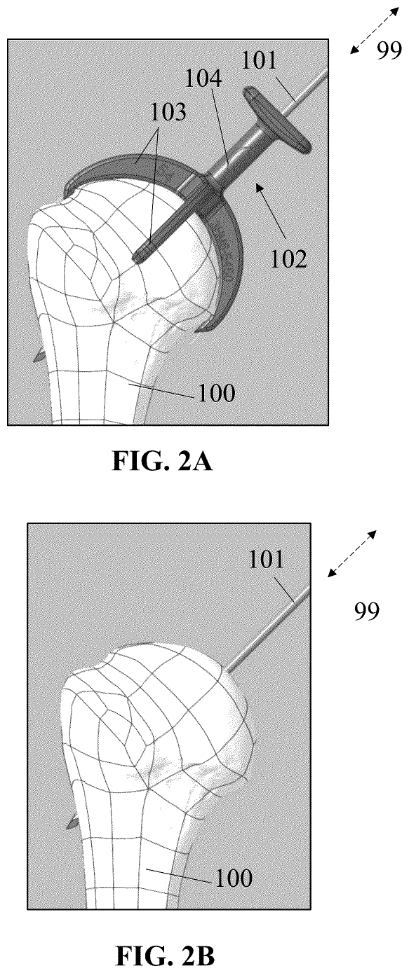

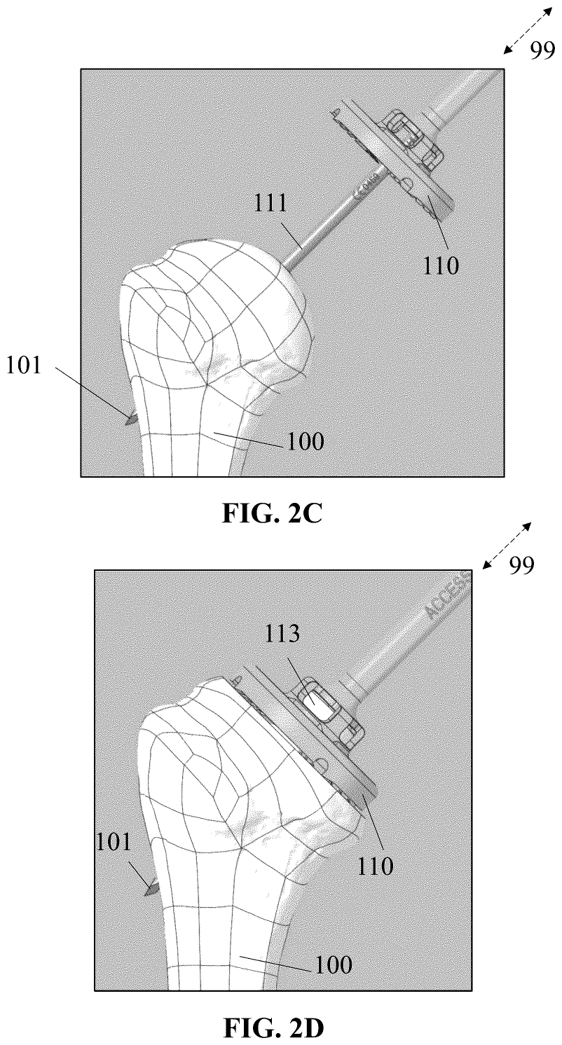

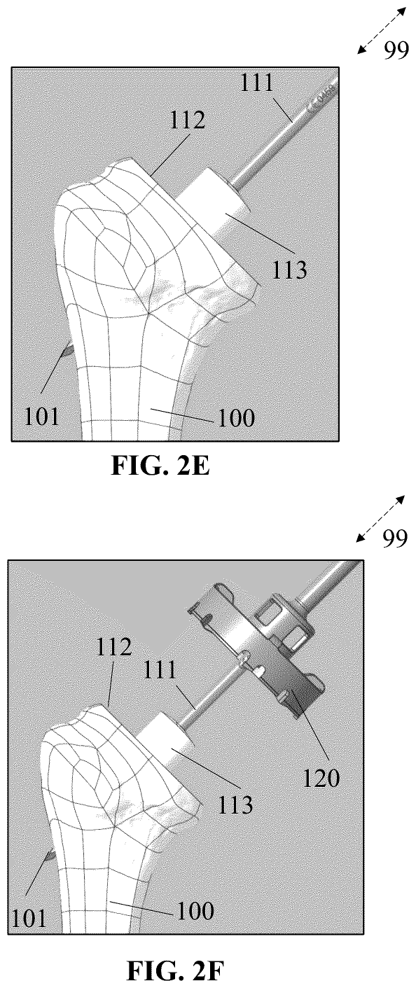

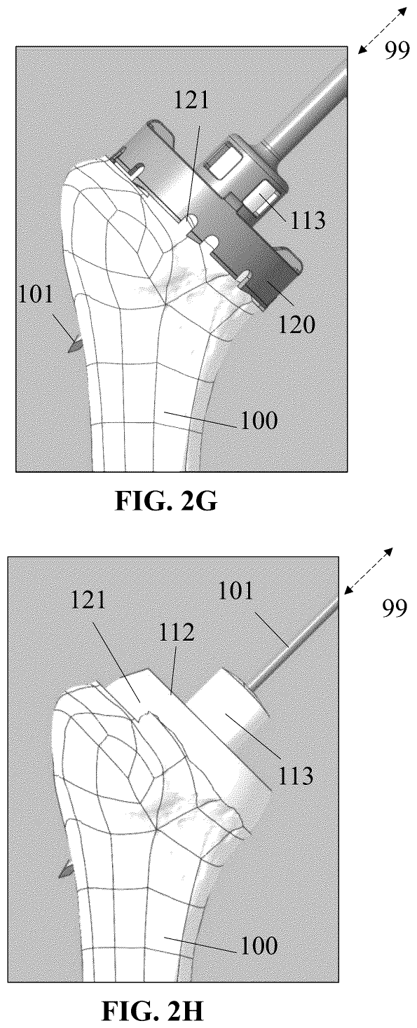

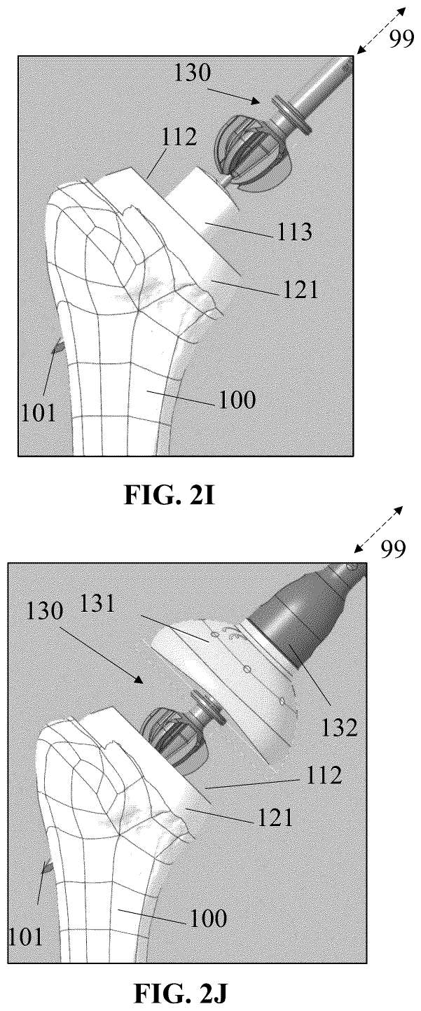

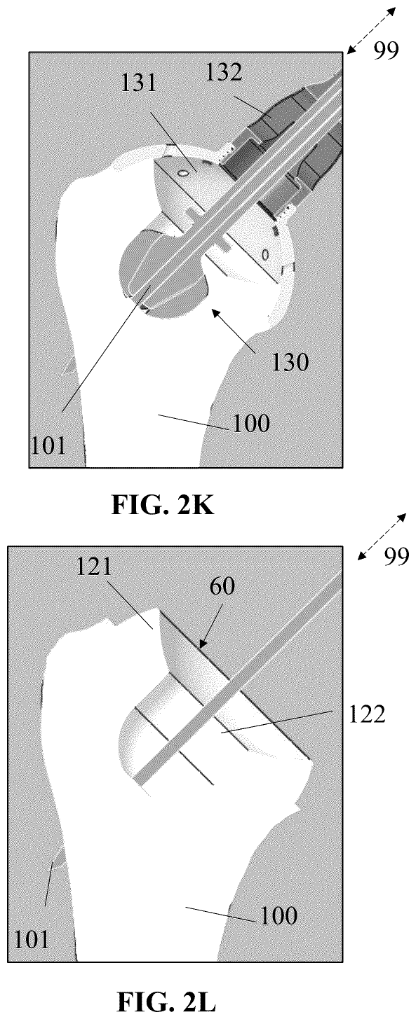

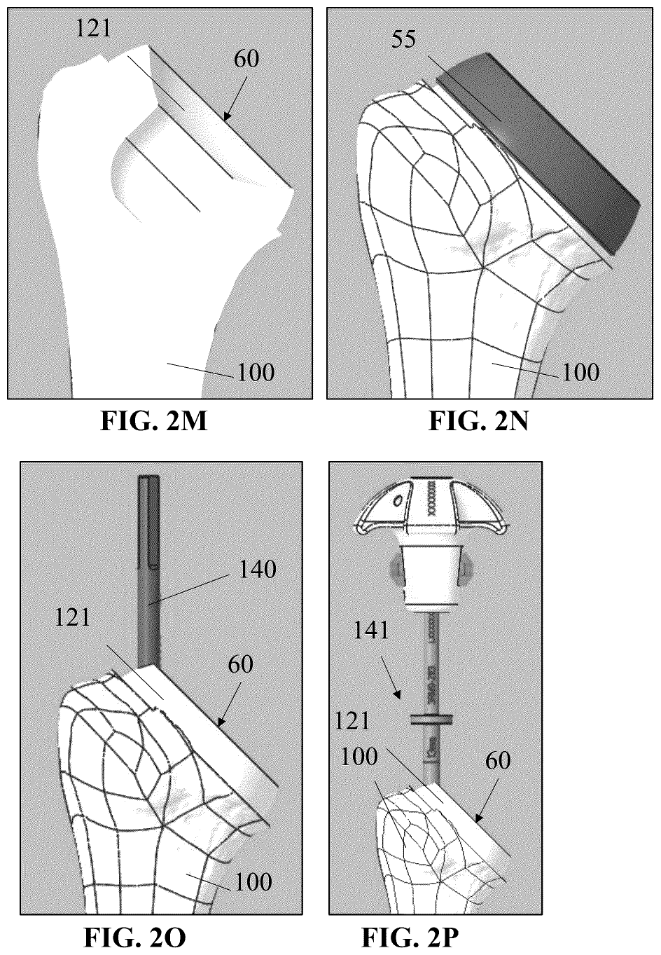

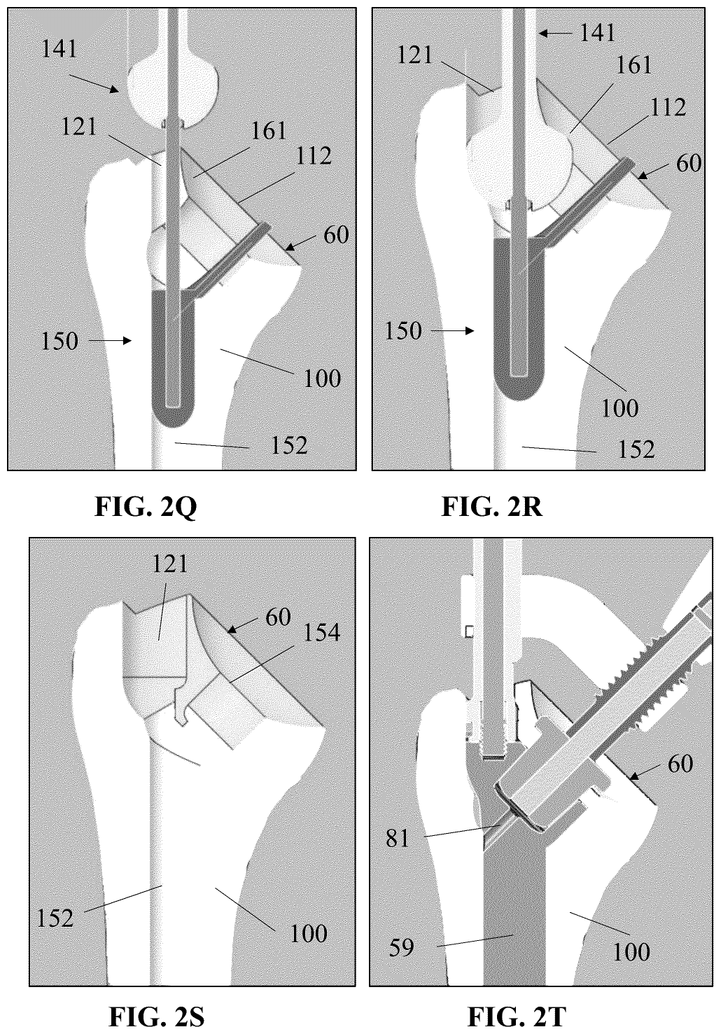

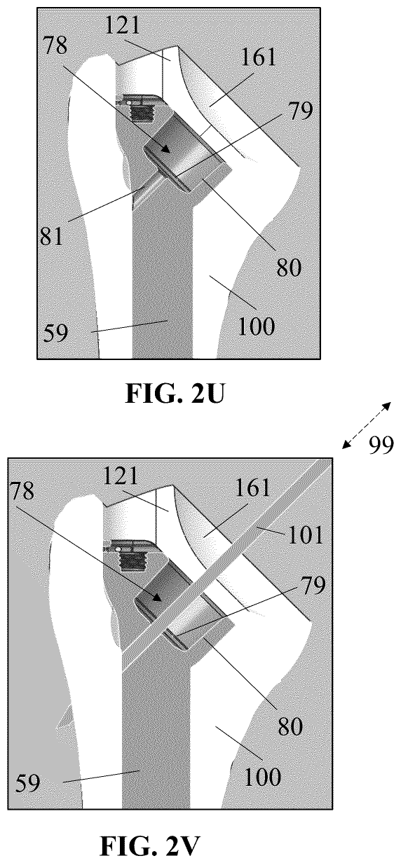

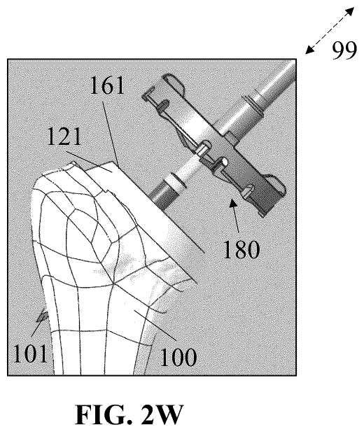

FIG. 1 depicts at least a portion of an exemplary humeral implant. In some embodiments, the humeral implant is configured to be coupled to the humerus at an excision portion. As used herein, the terms “excision portion” may be used interchangeably with “implant site” or “excision site”. In some embodiments, as described herein, the humeral implant site is located at proximal portion of the humerus (e.g., see FIGS. 2A-2H herein). In some embodiments, the excision portion includes a concave shaped inner implant site portion (e.g., see reference character 54), and an outer implant site surface (e.g., see reference character 58). In some embodiments, the outer an implant site surface resembles a ring shape and encircles the inner concave portion. In some embodiments, the inner concave portion defines an implant site opening (e.g., at a bottom portion of the inner implant site portion) providing access to a humeral stem portion (as described herein). As described herein, the bottom portion of the inner concave portion of the implant site may refer to a distal portion of the inner concave portion of the implant site interchangeably. FIG. 1 illustrates an exploded view of a reverse shoulder system and comprising a humeral implant 57 and a glenoid implant 350. In some embodiments, a humeral implant 57 is at least partially disposed in a humerus bone 100. In some embodiments, the humeral implant 57 comprises a liner 68, an implant tray 56, an adaptor 66, and a stem 59. In some embodiments, the implant tray 56 comprises a tray body portion 71, a tray lip portion 72, and a tray base portion 73. In some embodiments, the adaptor 66 comprises an adaptor wall 76 and an adaptor channel 67. In some embodiments, the stem 59 is at least partially disposed in a humerus 100. In some embodiments, a liner 68, an implant tray 56, an adaptor 66, and a stem 59 are configured to mate together.

In some embodiments, a glenoid implant 350 is configured to be at least partially disposed in a glenoid portion of a scapula bone 200 at a glenoid implant site 210. In some embodiments, a glenoid implant 350 comprises a glenosphere 310, a baseplate 272, and a glenoid fixation screw 260. In some embodiments, the glenosphere 310, the baseplate 272, and the glenoid fixation screw 260 are configured to couple at the glenoid implant site 210.

FIGS. 2A-W illustrate methods, systems, and tools used to prepare and place a humeral implant in a shoulder surgery (e.g., reverse shoulder surgery). In some embodiments, when placing a humeral implant 57 in a patient, a working axis 99 is established that corresponds to a proper angle of insertion (of the humeral implant) into a humerus 100. As shown in FIG. 2A, a working axis 99 may be designated by disposing a surgical guide pin 101 into a humerus 100 along the working axis 99. The surgical guide pin 101 may serve as a landmark to the surgeon for where the working axis 99 is located through the humerus 100. A surgical guide pin 101 may be inserted into a humerus 100 using a pin guide alignment device 102 to ensure the longitudinal axis of the surgical guide pin 101 passes through a working axis 99. In some embodiments, a pin guide alignment device 102 may have one or more pin guide alignment device arms 103 extending radially from the longitudinal axis of a pin guide alignment device body portion 104. In some embodiments, a pin guide alignment device 102 has four pin guide alignment device arms 103. In some embodiments, one or more pin guide alignment device arms 103 are curved towards a humerus 100 epiphysis. In some embodiments, one or more pin guide alignment device arm(s) 103 are configured to mate with the proximal articulating surface of a humerus 100 (e.g., at least a portion of the humeral head). A pin guide alignment device 102 may have a bore (not shown) extending through a longitudinal axis of pin guide alignment device body portion 104. In some embodiments, the bore may be configured to permit a surgical guide pin 101 to pass through the bore therein. In some embodiments, a surgical guide pin 101 passes through a bore disposed in a pin guide alignment device 102 and into a humerus 100 along a working axis 99. In some embodiments, the surgical pin guide is inserted into the humerus 100 using a drill, a hammer, or other device as known in the art.

As shown in FIG. 2B, once a surgical guide pin 101 has been inserted into a humerus 100, a pin guide alignment device 102 may be removed by sliding the pin guide alignment device 102 away from the articulating surface of a humerus 100 along a working axis 99 until it is free from the surgical guide pin 101.

A flat reamer 110 may have an opening disposed through a longitudinal axis (not shown). In some embodiments, the flat reamer 110 is advanced along a working axis 99 towards a humerus 100 such that a surgical guide pin 101 passes through the opening in the flat reamer 110. In some embodiments, such as illustrated in FIG. 2C, the flat reamer 110 is advanced along a working axis 99 towards a humerus 100 such that a centering shaft 111, disposed around a surgical guide pin 101, passes through the opening in the flat reamer 110. In some embodiments, a centering shaft 111 may be at least partially inserted into a humerus 100 and have a longitudinal axis extending along a working axis 99. In some embodiments, the centering shaft may include threads allowing a shaft to be threaded into the humerus.

As illustrated in FIG. 2D, a flat reamer 110 may interface with the articulating surface of a humerus 100 and rotate to ream a humerus 100 to form a flat bone surface 112 (not shown in 1D) and an inner bone cylinder 113. In some embodiments, the inner bone cylinder is formed based on a stopper function (not shown) of the flat reamer, thereby restricting an amount of bone removed from the portion of the humerus. The flat reamer 110 may be removed from the surgical area by sliding away from a humerus 100 along a working axis 99 and over a centering shaft 111 encircling a surgical guide pin 101. As shown in FIG. 2E after the flat reamer 110 has reamed the articulating surface of a humerus 100, the articulating surface has a flat bone surface 112 of bone disposed generally perpendicular to working axis 99. In some embodiments, the resulting humeral surface after reaming with the flat reamer 110 has an inner bone cylinder 113 extending in a proximal direction away from an inner portion of a flat bone surface 112 in a substantially cylindrical shape. In some embodiments, the longitudinal axis of an inner bone cylinder 113 is aligned with a working axis 99. In some embodiments, an inner bone cylinder 113 may have an outer diameter smaller than a flat bone surface 112 outer diameter.

As shown in FIG. 2F a concave reamer 120 may be passed over a centering shaft 111 along a working axis 99 and towards a flat bone surface 112. As shown in FIG. 2G a concave reamer 120 may rotate and ream at least a portion of an outer surface of a humerus 100 extending distally from a flat bone surface 112. In some embodiments, a concave reamer 120 reams the external surface of a humerus 100 to form a circumferential bone surface 121. In some cases, the circumferential bone surface corresponds to the outer surface of the excision or implant site 60, as described herein. In some embodiments, the concave reamer 120 further reams an interior surface of the humerus, thereby forming an inner concave surface. In some embodiments, said inner concave surface corresponds to the inner concave surface of the implant site, as described herein. As illustrated in FIG. 2H, a concave reamer 120 may be removed by sliding it along a working axis 99 away from a flat bone surface 112 and over a centering shaft 111 encircling a surgical guide pin 101. In some embodiments, the concave reamer 120 includes a stopper so as to limit the amount of bone removed from the humerus. In some embodiments, a circumferential bone surface 121 has a convex curved shape. In some embodiments, a centering shaft 111 is removed after a concave reamer 120 has reamed a humerus 100.

As illustrated in FIG. 2I, an angled reamer 130 may be passed over a surgical guide pin 101 towards humerus implant site. As shown in FIG. 2J, an angled reamer 130 may ream a humerus 100 from a proximal end of an inner bone cylinder 113 and distally to an inner surface of the implant site of the humerus (e.g., bounded by a circumferential bone surface 121). In some embodiments, the angled reamer 130 forms an opening 122 at a distal end of the inner concave surface of the implant site. In some embodiments, an angled reamer 130 may ream a humerus 100 at least partially to a spongy bone region of an epiphysis, a medullary cavity, or a combination of spongy bone and medullary cavity of the humerus 100. As illustrated in FIG. 2J, a first handle portion 131 connected to a distal end of a second handle portion 132 may be passed over a surgical guide pin 101 and an angled reamer 130, wherein a surgical guide pin and a shaft for the angled reamer are configured to pass through an opening of the first and second handle portion. A first handle portion 131 may have a concave curved outer surface. As shown in FIG. 2K a first handle portion 131 may be configured to fit over a circumferential bone surface 121. As shown in FIG. 2L, an angled reamer 130, a first handle portion 131, and a second handle portion 132 may be removed from the surgical field by sliding the members in a direction away from a humerus along a working axis 99. In some embodiments, a humerus channel 152 extends from an opening 154 formed in the inner concave surface 161 of the implant site 60 to the medullary cavity.

As illustrated in FIG. 2M, a surgical guide pin 101 may be removed after an angled reamer 130, a first handle portion 131, and a second handle portion 132 have been removed from the surgical field leaving a reamed humerus 100. As shown in FIG. 2N a trial tray 55 may be fit over a circumferential bone surface 121. As shown in FIG. 2O, a trial tray 55 may be removed and a pilot hole may be drilled into the proximal surface of a humerus 100 with a drill 140. The drill 140 may be removed once a pilot hole is disposed in a humerus 100. As shown in FIG. 2P, a stem reamer 141 may be used to ream the pilot hole (not shown) in the humerus 100. As shown in FIGS. 2Q-2T a canal guide 150 can be inserted within a humerus 100 and used to guide a stem reamer 141 into a proximal surface of the humerus 100. In some embodiments, the opening created by a stem reamer 141 is configured to permit a stem 59 to pass therethrough (which may correspond to stem 59 as described herein).

As depicted in FIGS. 2U and 2V once a stem 59 has been placed in a humerus 100, a surgical guide pin 101 may be inserted along a working axis 99 through an inner concave surface 161 and a stem 59. In some embodiments, a stem 59 has a stem cavity 78 disposed in an implant facing surface. In some embodiments, a stem cavity 78 is generally cylindrical in shape. In some embodiments, a stem cavity 78 is generally oval, square, rectangular, pentagonal, hexagonal, heptagonal, octanol, nonagonal, or combinations thereof, in shape. In some embodiments, a stem 59 has a stem bore 81 extending through a working axis 99. A stem bore 81 may pass through a longitudinal axis of a stem cavity 78. In some embodiments, a diameter of a stem bore 81 is smaller than a diameter of a stem cavity 78. In some embodiments, a diameter of a stem bore 81 is configured to receive a surgical guide pin 101 therein. In some embodiments, a stem 59 is disposed at least partially within a humerus 100. In some embodiments, a stem 59 is disposed at least partially within a humerus 100 and secured at least partially by coupling with an adaptor 66, an implant tray 56, a liner 68, or combinations thereof, as described herein. In other cases, the stem 59 is further secured, or alternatively secured, at least partially with bone cement and/or threading (e.g., on an outer surface of the stem).

As shown in FIG. 2W, a cleanup reamer 180 may be passed over a surgical guide pin 101 along a working axis 99 towards a curved bone surface 161 of a humerus 100. A cleanup reamer 180 may have a bore extending through the longitudinal axis. A bore disposed in a cleanup reamer 180 may be configured to have a larger diameter than a surgical guide pin 101. A cleanup reamer 180 may be removed from the implant site by sliding the cleanup reamer 180 away from an inner concave surface 161 along a working axis 99 until the cleanup reamer 180 no longer has a surgical guide pin 101 disposed within the cleanup reamer 180 bore. In some embodiments, a cleanup reamer 180 is configured to ream an inner concave surface 161 until the rim of an inner concave surface 161 is substantially perpendicular to a working axis 99. In some embodiments, the cleanup reamer 180 is configured to ream the proximal end of the humerus bone 100 to ensure it is true and properly aligned with the stem 59.

Different bones within the body are used as foundations to affix orthopedic implants. Bones in the body have different amounts of cortical and cancellous or trabecular bone depending on the mechanical load and articulation. Therefore, bone density and relative bone hardness varies from bone to bone. In order to place to affix an orthopedic implant to a bone, a bore is generally drilled into the bone and at least a portion of the orthopedic implant is placed into the bore to fix the implant into place.

If the portion of the bone the bore is drilled into is unhealthy, demineralized, low in bone density, or soft, there can be insufficient support or surface area to hold the orthopedic implant in place and support mechanical load and articulation of a joint.

Traditional orthopedic reamers drill into bone using a drill head with angular threads configured to remove bone particles from the bore. Described herein is a reamer designed to compact bone particles along the internal surface of the bore to enhance mechanical strength and support of an implant.

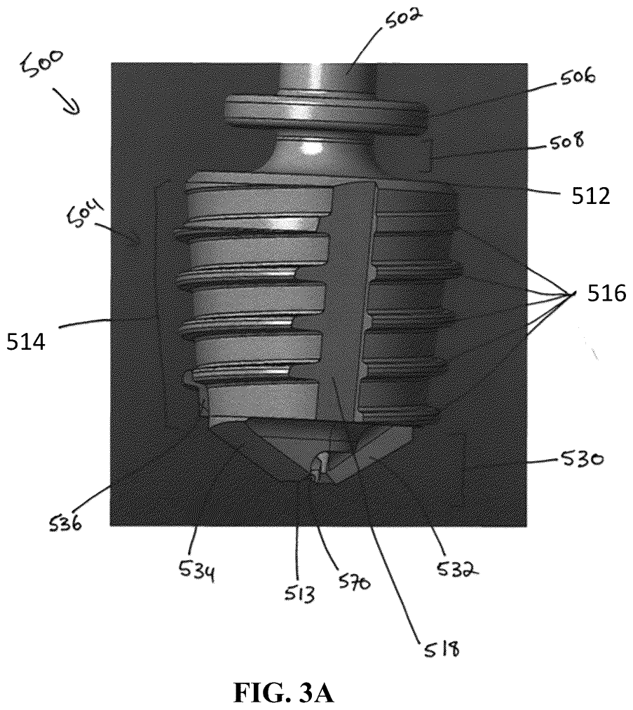

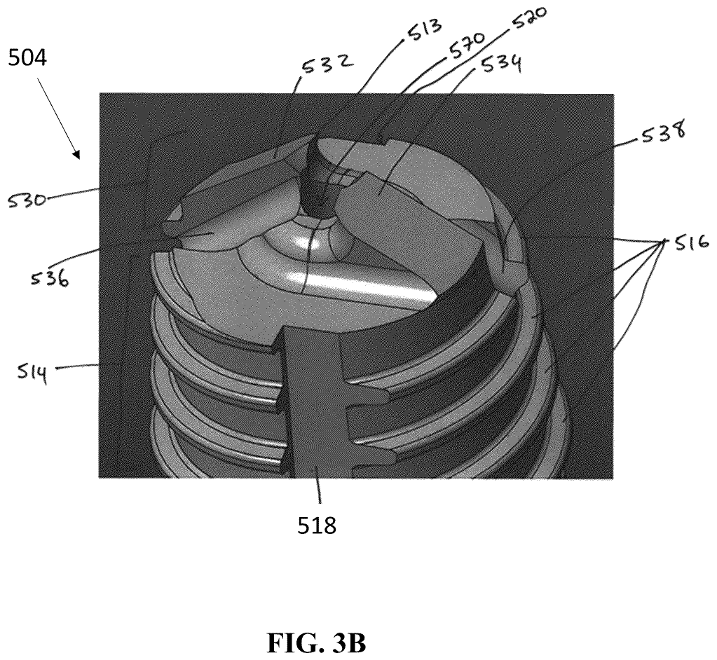

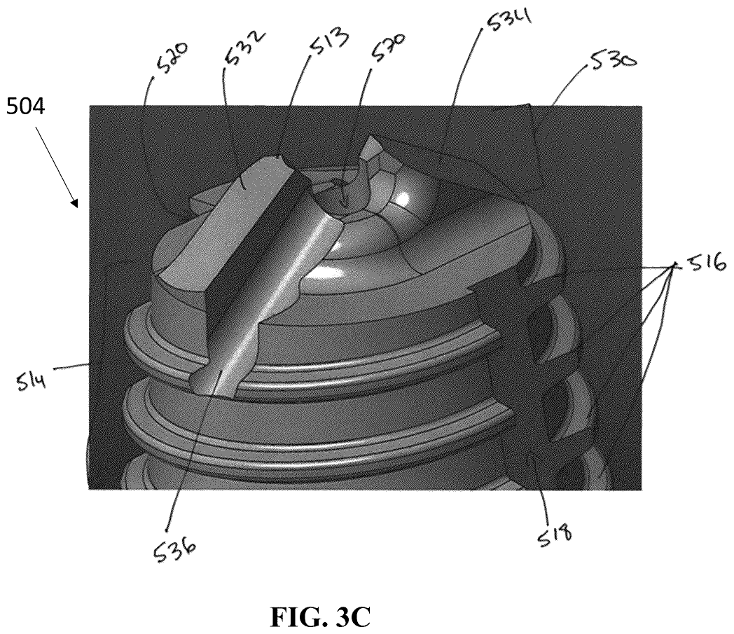

FIG. 3A illustrates a reamer 500 having a shank 502 and a cutting body 504 according to certain embodiments described herein. In some embodiments, the shank 502 may extend from a handle (not shown) to a cutting body 504. The distal end of the shank 502 may have an annular disk 506 extending radially from the longitudinal axis of the shank 502. In some embodiments, the annular disk 506 is configured to act as a stop for drilling. For example, the annular disk 506 may be configured to stop further drilling when a depth has been reached that causes the annular disk 506 to interface with the external surface of a bone. The distal end of the annular disk 506 may be attached to a neck region 508 connecting the shank 502 to the cutting body 504. In some embodiments, the neck region 508 is curved inwards towards the longitudinal axis of the reamer 500. In some embodiments, the shank 502 may be releasably attached to the cutting body 504. In some embodiments, the shank 502 and the cutting body 504 are one component.

The cutting body 504 may extend from a proximal cutting body end 512 to a distal cutting body end 513. The cutting body 510 may have a threaded region 514 extending from the proximal cutting body end 512 to a tapered cutting region 530. The threaded region 514 may have one or more threads 516 extending radially about the longitudinal axis of the cutting body 504. In some embodiments, the threads 516 are angled 0°, 5°, 10°, 15°, 20°, 25°, or 30°, relative to the circumference axis of the threaded region 514. In some embodiments, the threads 516 are configured to retain and compact bone particles about the internal surface of a bone bore. In some embodiments, the threads 516 are blunted or rounded in shape. In some embodiments, a first longitudinal notch 518 is disposed into the cutting body 504 in the threaded region 514 about the longitudinal axis. In some embodiments, a second longitudinal notch 520 is disposed into the cutting body 504 in the threaded region 514 about the longitudinal axis (see FIGS. 3B and 3C). In some embodiments, at least one longitudinal notch forms a cavity in the threaded region having a semicircular, triangular, square, or any other shape known to those skilled in the art. In some embodiments, at least one of the longitudinal notches 518 and 520 is configured to prevent bone particles from exiting the bore. In some embodiments, at least one of the longitudinal notches 518 and 520 are configured to contact the compaction of bone against the inner surface of the bone bore.

The tapered cutting region 530 may extend from the threaded region 514 to the cutting body distal end 513. The tapered cutting region 530 may be tapered from the threaded region 514 to the cutting body distal end 513. The tapered region 530 may have a first protrusion 532 extending from the external surface of the tapered region 530. In some embodiments, the tapered region 530 may have a second protrusion 534 extending from the external surface of the tapered region 530. At least one of the protrusions 532 and 534 may extend in a substantially rectangular shape. At least one of the protrusions 532 and 534 may be configured to cut into bone to form a bore in the bone. In some embodiments, the tapered region has a first cutting channel 536.

FIGS. 3B and 3C illustrate various angles of the cutting body 504 according to embodiments described herein. FIG. 3B illustrates a portion of the threaded region 514 and the tapered cutting region 530. The tapered cutting region 530 may have a first channel 536 extending from the cutting body distal end 513 to the threaded region 514. The tapered cutting region 530 may have a second channel 538 extending from the cutting body distal end 513 to the threaded region 514. In some embodiments, at least one of the first and second channels 538 are configured to move bone particles created from reaming from the cutting region to the internal surface of the bone bore.

As shown in FIGS. 3B and 3C, an opening 570 may be disposed in the cutting body distal end 513. In some embodiments, the opening extends alone the longitudinal axis of the reamer 500 from the cutting body distal end 513 through the handle (not shown) of the reamer 500. In some embodiments, the opening 570 is configured to have a diameter wide enough to allow a surgical guide pin to pass therethrough. In some embodiments, the reamer 500 is used as described herein relating to reamers 141, 130, 120, 110, and/or 180.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A reamer for performing an orthopedic surgery on a subject, the reamer comprising:
a handle configured to be gripped by a user; and
a cutting body coupled to the handle and comprising
i) a threaded region configured to compact bone particles within a bone bore, and
ii) a tapered region configured to cut a bore into bone.

2. The reamer of claim 1, further comprising a shank configured to couple the handle to the cutting body.

3. The reamer of claim 2, wherein the shank is releasably coupled with the cutting body.

4. The reamer of claim 2, further comprising an annular disk extending radially from the shank.

5. The reamer of claim 4, wherein the annular disk is spaced apart from the tapered region so as to correspond to a maximum depth of the tapered region within the bore when the annular disk interfaces with an outer surface of the bone.

6. The reamer of claim 2, further comprising a neck region extending between the shank and the cutting body, the neck region tapering radially inwards from a wide end interfacing with the cutting body to a narrow end interfacing with the shank.

7. The reamer of claim 1, wherein the tapered region further comprises a first protrusion extending from an external surface of the tapered region.

8. The reamer of claim 7, further comprising a second protrusion extending from the external surface of the tapered region.

9. The reamer of claim 1, wherein the tapered region further comprises a first cutting channel.

10. The reamer of claim 1, further comprising an opening extending through a longitudinal axis of the cutting body.

11. The reamer of claim 1, wherein the threaded region further comprises a first notch extending longitudinally between a proximal and distal end of the cutting body and at least partially recessed into the threaded region.

12. The reamer of claim 11, wherein the threaded region further comprises a second notch extending longitudinally between the proximal and distal end of the cutting body and at least partially recessed into the threaded region.

13. The reamer of claim 1, wherein the threaded region comprises one or more threads angled 0°, 5°, 10°, 15°, 20°, 25°, or 30°, relative to a circumference axis of the threaded region.

14. A method of forming a bore in a bone; the method comprising:
reaming a portion of the bone to form the bore within the bone;
compacting a plurality of bone particles against an inner surface of the bore by rotating a cutting body comprising a threaded region and a tapered region, wherein the threaded region comprises one or more threads configured to compact the plurality of bone particles about the inner surface of the bone bore; and
removing the cutting body from the bone bore.

15. The method of claim 14, wherein the one or more threads are angled 0°, 5°, 10°, 15°, 20°, 25°, or 30°, relative to a circumference axis of the threaded region.

16. The method of claim 14, wherein the cutting body is releasably coupled to a shank.

17. The method of claim 16, further comprising reaming the bore to achieve a final depth of the tapered region within the bore, such that an annular disk extending radially from the shank interfaces with an outer surface of the bone.

18. The method of claim 14, wherein the threaded region further comprises a first notch extending longitudinally between a proximal and distal end of the cutting body and at least partially recessed into the threaded region.

19. The method of claim 18, wherein the threaded region further comprises a second notch extending longitudinally between the proximal and distal end of the cutting body and at least partially recessed into the threaded region.

20. The method of claim 14, further comprising passing an opening extending through a longitudinal axis of the cutting body over a surgical guide pin disposed in the bone.
